(19) United States
(12) Patent Application Publication
(10) Pub. No.: US 2007/0078350 A1
(43) Pub. Date: Apr. 5, 2007

(54) Laser imaging apparatus with variable power, orbit time and beam diameter

(76) Inventors: Robert H. Wake, Cooper City, FL (US); Steven L. Ponder, Ft. Lauderdale, FL (US); Gary M. Becker, Boca Raton, FL (US)

Correspondence Address: SHLESINGER, ARKWRIGHT & GARVEY LLP, SUITE 600, 1420 KING STREET, ALEXANDRIA, VA, 22314, US

(21) Appl. No.: 11/542,642

(22) Filed: Oct. 4, 2006

Related U.S. Application Data
(60) Provisional application No. 60/723,004, filed on Oct. 4, 2005.

(51) Int. Cl. A61B 5/05; A61B 6/00

(52) U.S. Cl. 600/476; 600/407; 600/426

(57) ABSTRACT

An apparatus for breast scanning comprises a patient support for a patient to rest in a prone position, the support having an opening with one of her breasts vertically pendent through the opening for scanning; and a laser CT scanner disposed below the support for generating data for reconstruction of images of the breast. The laser CT scanner includes a laser beam for impinging on the breast. The laser beam is orbitable around the breast. The laser CT scanner includes a plurality of detectors positioned in an arc around the breast to simultaneously detect light transmitted through the breast. The measured signal level at the detectors is maintained to an acceptable level while controlling the temperature rise on the breast surface during scanning.

### RELATED APPLICATION

This is a nonprovisional application claiming the priority benefit of provisional application Ser. No. 60/723,004, filed Oct. 4, 2005, incorporated herein by reference.

### FIELD OF THE INVENTION

The present invention is generally directed to optical imaging apparatus and in particular to laser CT scanners for imaging breasts.

### BACKGROUND OF THE INVENTION

The attenuation of light through the breast in an optical tomographic scanner is very large, as high as 10⁷:1. The typical optical CT scanner geometry, as described in U.S. Pat. No. 5,692,511, is illustrated in FIGS. 1 and 2, where a light source 10, typically a near-infrared laser, illuminates the scanned object, typically a breast 6. A ring of detectors 12 views the scanned object, each detector seeing light that is transmitted through a portion of the breast and re-emitted. For several detectors, the light paths 16, 18 and 20 are shown.

The light levels at the detectors are generally quite low and vary with detector position and scanned object size and composition. The light transmission is given by:
I=I₀ e⁻^{^{μ}}^{x} Equation 1:
where I is the detected intensity, I₀ is the incident intensity, μ is the effective linear attenuation coefficient of the medium and x is the path length in the medium. For a μ of 1.0 cm⁻¹, a typical value for tissue, and path lengths of 20 cm, the detected intensity I is on the order of 10⁻⁸ times the incident intensity I₀.

Exacerbating the light detection problem is the fact that the scattering in the breast causes the light to be emitted from the entire surface of the breast, even though only a several millimeter area is being illuminated. This scattering causes another reduction of intensity by a factor of 10³ to 10⁴. The net effect is that a detector receiving light from a small (several millimeter) area on the surface of the breast will see, in the worst case, a light signal that is 10⁻¹¹-10⁻¹² times the incident light intensity.

The signal detected is the detected light intensity times the measurement time, namely the total number of light photons collected. The measurement time is proportional to the total rotation time of the scanning mechanism, since a certain minimum number of measurements must be taken during one rotation in order to perform the computed tomographic image reconstruction. Typically 100-200 measurements must be taken per detector in each revolution in order to reconstruct an image of that section of the breast. So for a given patient (a given μ) and given breast diameter (x) at the level of the laser and detectors, the measured signal is given by:
S≡PT Equation 2:

where: P is the laser power in Watts
T is the rotation time of the scanning mechanism
   The measured signal is directly proportional to the laser power and to the scanning mechanism rotation time.

Compounding this measurement problem is the need to perform the scan in a minimum of time, for reasons of patient comfort and economic return to the institution performing the scan.

Increasing the incident power of the laser will increase the measured signals proportionately, but a large fraction of this laser power is absorbed, converted to heat at the point that the laser is incident on the breast. This energy will cause heating of the skin and tissue immediately under the skin. And excessive heating will cause pain and ultimately will cause tissue damage and destruction.

The temperature rise of tissue briefly irradiated by a laser is given by:

$$Δ T = \frac{{μ}_{a} H}{ρC} \qquad Equation 3 \text{:}$$

where: ΔT is the tissue temperature rise in ° C.
   μₐ is the tissue absorption coefficient in cm⁻¹
   H is the radiant flux in Joules/cm²
   ρ is the tissue density in g/cm3
   C is the tissue specific heat in J/g° C.

In the scanning geometry of FIGS. 1 and 2, the laser beam passes over an area of tissue as the scanning mechanism rotates. The radiant flux is given by:

$$H = \frac{4 PT}{{π}^{2} dD} \qquad Equation 4 \text{:}$$

where: H is the radiant flux in Joules/cm²
   P is the laser power in Watts
   T is the rotation time of the scanning mechanism
   d is laser beam diameter in cm
   D is the diameter of the breast at the level of the laser

For any given patient, the μₐ, ρ and C are constants. Thus the temperature rise is given by:

$$Δ T ∝ \frac{PT}{dD} \qquad Equation 5 \text{:}$$

The temperature rise is directly proportional to the laser power and the rotation time and is inversely proportional to the laser spot diameter and the breast diameter at the plane of the scan.

As an example, a 500 milliwatt laser collimated to a 3.0 mm diameter beam rotating in 10 seconds around a 5 cm diameter breast with very darkly pigmented skin (μₐ=40 cm⁻¹) will cause a temperature rise of 5.3° C. Any transient temperature rise less than 10° C. is not harmful and is likely not perceptible by the patient.

### OBJECTS AND SUMMARY OF THE INVENTION

It is an object of the present invention to provide a breast scanning apparatus and method that maintains the measured signal level at the detectors to an acceptable level while controlling the temperature rise of the surface of the breast being scanned by adjusting one of the laser power, beam spot diameter and orbit time of the laser beam depending on the breast diameter at a scan plane.

It is another object of the present invention to provide a breast scanning apparatus and method that reduces the scan time by increasing the laser power and increasing the rotation rate of the scanner (decreasing the time per orbit) while controlling the temperature rise of the surface of the breast being scanned.

It is still another object of the present invention to provide a breast scanning apparatus and method that changes one of the laser power, beam spot diameter and orbit time of the laser beam during the scan as the breast diameter changes at the level of the laser beam (scan plane) in such a way that the temperature rise on the surface of the breast is controlled.

In summary, the present invention provides an apparatus for breast scanning comprising a patient support for a patient to rest in a prone position, the support having an opening with one of her breasts vertically pendent through the opening for scanning; and a laser CT scanner disposed below the support for generating data for reconstruction of images of the breast. The laser CT scanner includes a laser beam for impinging on the breast. The laser beam is orbitable around the breast. The laser CT scanner includes a plurality of detectors positioned in an arc around the breast to simultaneously detect light transmitted through the breast. The measured signal level at the detectors is maintained to an acceptable level while controlling the temperature rise on the breast surface during scanning.

The present invention also provides a method for scanning a breast, comprising: a) positioning a patient in a prone position on a support having an opening with one of her breasts vertically pendent through the opening; b) scanning the breast with a laser CT scanner with a laser beam orbiting around the breast; d) detecting with a plurality of detectors positioned in an arc around the breast the light transmitted through the breast; e) determining the perimeter of the breast; and f) decreasing the orbit time as the diameter of the breast at scanning planes decreases, thereby reducing the scan time for the breast.

### BRIEF DESCRIPTION OF THE DRAWINGS

FIG. 1 is a schematic side elevational view of a laser imaging apparatus with a patient in a prone position with one of her breasts positioned within a scanner for an optical tomographic study.

FIG. 2 is a schematic top view of an optical scanner of FIG. 1, showing the breast disposed within an arc of detectors.

FIG. 3 is a schematic perspective view, showing an arrangement for helical orbital movement of the laser beam and detectors shown in FIG. 2.

FIG. 4 is a schematic diagram of a frequency synthesizer for controlling a stepping motor shown in FIG. 3.

FIG. 5 is a schematic diagram of a computer-controlled laser system.

FIG. 6 is a graph of a laser output power versus laser drive current.

FIG. 7 is a schematic diagram of a focal zoom lens assembly for controlling the laser beam spot size.

### DETAILED DESCRIPTION OF THE INVENTION

The present invention addresses the issue of increasing the throughput of a laser scanning system by increasing the laser power and proportionally decreasing the rotation time of the scanner, while maintaining the measured signal quality. The present invention further discloses modifying the laser power and/or rotation time and/or laser beam spot size as the breast diameter changes during the scan. This is done while advantageously controlling the temperature rise on the surface of the breast during the scan to an acceptable level.

As the system scans the breast, starting typically at the chest wall and progressing towards the nipple, the breast diameter D in Equation 5 at the level of the laser beam and the detectors will generally get smaller. Breasts are not necessarily circular in cross-section, but an approximation as a circle is sufficient for estimating heating. A circumscribing circular diameter or a circle with the same area or perimeter length as the actual cross-section are reasonable approximations. The minimum breast diameter D is intended to be as small as possible, so that most of the breast, approaching the nipple, can be scanned without excessive heating. A D of a few centimeters is typical. Thus the variables that can be controlled are the laser power P, the rotation time T and the laser beam diameter d. The breast perimeter is measured during the scan as disclosed in U.S. Pat. Nos. 6,029,077 and 6,044,288.

Current laser scanning systems employ lasers of up to 500 milliwatts power at wavelengths from 650-950 nanometers, the “tissue window” where tissue exhibits relatively low attenuation of light. The rotation times are from 20-30 seconds per slice (scan plane). Typically 20-40 slices are acquired in the scan of a breast, leading to scan times of typically 10- 20 minutes. Laser beam spots are 2-4 millimeters in diameter.

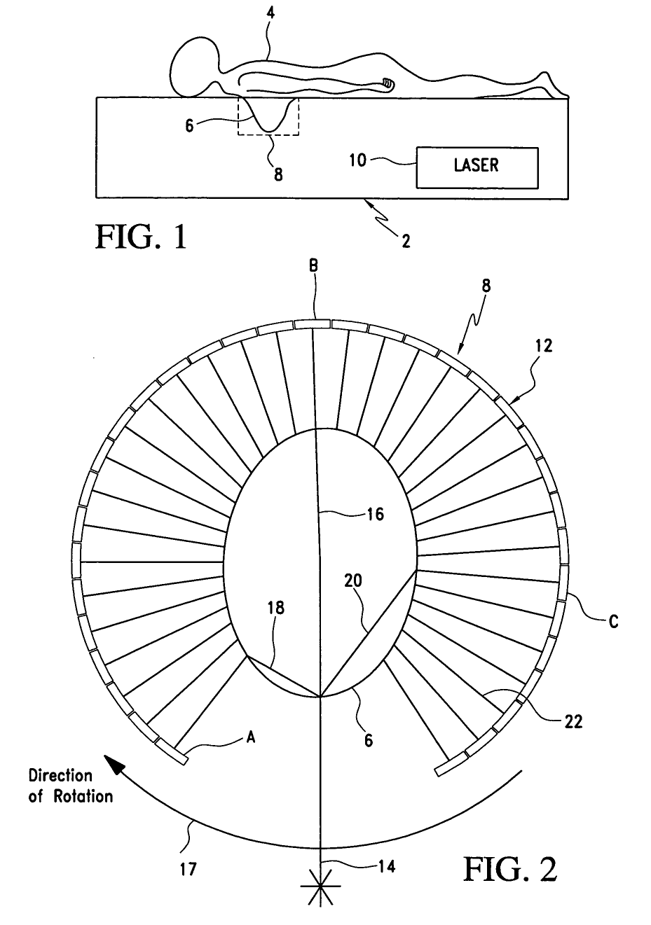

An optical tomographic scanning apparatus 2, such that disclosed in U.S. Pat. No. 5,692,511, is schematically shown in FIG. 1. A patient 4 is positioned prone on a top surface of the apparatus 2 with her breast 6 disposed pendant through an opening of the top surface so as to be within an optical scanner 8. A laser beam from a laser source 10 is brought to the scanner 8 to illuminate the breast 6.

The optical scanner 8 comprises a detector ring 12 disposed around the breast in an arc, as shown in FIG. 2. A laser beam 14 impinges on the breast 6 creates a beam spot on the breast surface. The laser beam traversing through the breast and exiting at the other side, as generally disclosed at 16, 18 or 20, is picked up by the respective detectors A, B and C. The laser beam 14 and the detector ring 12 are orbited around the breast for a complete circle in the direction generally indicated at 17. At each angular position in the orbit, light detected by the detector ring 12 is recorded for later use in reconstructing an image of the breast 6.

The preferred photodetector for the optical scanner 8 is a silicon photodiode. Photodiodes exhibit small physical size and insensitivity to acceleration and magnetic fields, unlike photomultiplier tubes. A photodiode's quantum efficiency is far better than a photomultiplier's at the 800 nm near-infrared wavelength of biological interest. They are available with extremely small leakage currents for photoconductive application and high shunt resistances for photovoltaic application, and they are relatively inexpensive. Alternatively, avalanche photodiodes, photomultiplier tubes, microchannel plates or virtually any other form of optical detector could also be employed.

The laser beam 14, preferably a near-infrared laser, illuminates the breast and each detector sees light that is transmitted through a portion of the breast and re-emitted, such as for detectors A, B and C, for which light paths 18, 16 and 20 are shown for illustration purposes. Each detector has a restricted field of view axis as generally indicated at 22.

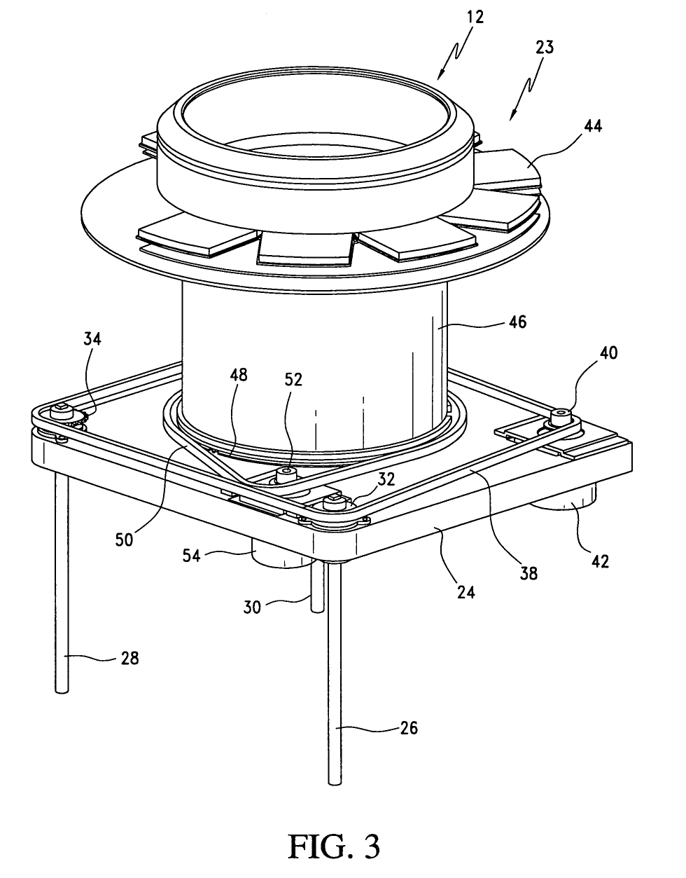

The optical scanner 8 of FIG. 2 is mounted on a helical scanning mechanism 23, as shown in FIG. 3. A helical scanning mechanism is the preferred approach for an optical CT scanner, where the orbital motion around the breast is continuous as is the elevator motion down the breast. The laser beam 14 and detectors 12 describe a helical path around the breast, akin to a screw thread. The helix pitch, the spacing of the detector and laser orbits, is typically 1-2 millimeters. Typically 50-100 orbits are required to scan the entire breast.

An elevator plate 24 is supported by and moves vertically on three ACME screws 26, 28 and 30. These three ACME screws are attached to a common baseplate at their bottom ends (not shown for clarity). In the preferred embodiment, the ACME screws do not rotate; rather the ACME nuts associated with the screws rotate. The ACME nuts are bonded to chain sprockets 32, 34 and a third sprocket 36 (hidden from view). The chain sprockets are connected by a roller chain 38 which is driven by sprocket 40 affixed to a stepping motor 42. Thus, the stepping motor 42 causes the elevator plate 24 to “crawl” up and down on the fixed ACME screws 26, 28 and 30 as it rotates. In the preferred embodiment, sprockets 32, 34 and 36 have 20 teeth, sprocket 40, 16 teeth and the ACME screws 26, 28 and 30 have a 4 millimeter lead. The stepping motor 42 is a 1.80° per full step motor, operated electrically at ⅛ stepping. Thus, each (⅛) step of stepping motor 42 will raise or lower elevator plate 24 and the detector ring 12 and the associated detector electronics 44 by 1/500 millimeter, or 2 microns. Typical elevator speeds are between 0.5 and 10 millimeters per second, or 250 to 5000 steps per second.

A rotating cylinder 46 is mounted on a ball bearing (not shown for clarity) attached to the elevator plate 24. It supports the detector ring 8 and detector electronics 44. A chain sprocket 48 is mounted on the base of the rotating cylinder 46 and is driven by roller chain 50, which is itself driven by sprocket 52 affixed to stepping motor 54. Thus, stepping motor 54 precisely controls the orbital position of the detectors in the detector ring 12 and detector electronics 44. In the preferred embodiment, sprocket 48 has 120 teeth, sprocket 52, 24 teeth and stepping motor 54 is a 1.80° per full step motor, operated electrically at ¼ stepping. Thus, each (¼) step of stepping motor 54 rotates the detector ring 12 and detector electronics 44 by 0.090°, 1/4000 of a 360° revolution. Typical orbit speeds are between 0.5 and 5 seconds per revolution or 800 to 8000 steps per second.

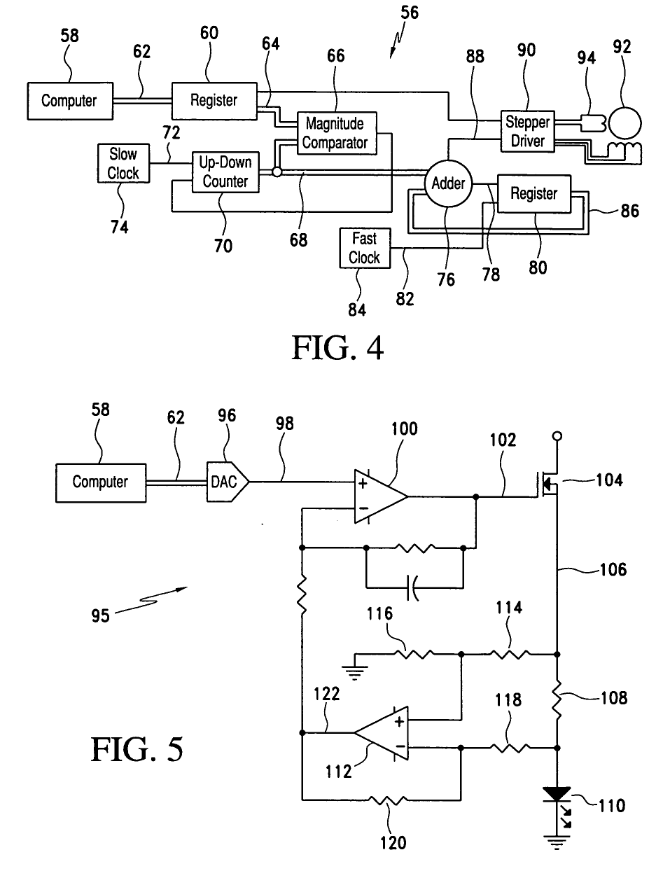

A schematic diagram of a frequency synthesizer 56, which provides the means for controlling each of the stepping motors 42 and 54, is disclosed in FIG. 4. A general purpose computer 50 loads a register 60 via an I/O bus 62. The value in the register 60 is a signed velocity value (speed and direction) (“move up at 3000 steps per second,” for example). The “requested” speed value 64 in register 60 is applied to a magnitude comparator 66, which compares the requested speed 64 to the actual speed 68. The actual speed 68 is the output of an up-down counter 70 which is clocked by a clock signal 72 generated by a slow clock generator 74. The behavior of the up-down counter 70 is determined by the output of the magnitude comparator 66 as follows:

if the actual speed equals the desired speed—do not count

if the actual speed is less than the desired speed—count up

if the actual speed is greater than the desired speed—count down

In this way, the actual speed signal 68 will be a trapezoid with linear rises and falls determined by the frequency of the slow clock 74. With a 1 kHz slow clock rate, if the computer 58 changes the desired rate 64 from 0 to 3000, the actual clock rate 68 will ramp from 0 to 3000 in 3 seconds and then maintain a value of 3000. This is advantageously done to limit the acceleration of the stepping motors so that the inertial loads can be accelerated by the motor's rated torque.

The actual clock rate 68 is applied to an adder 76. The adder's output 78 is stored by “phase” register 80, clocked by clock signal 82 generated by fast clock generator 84. The phase output 86 of register 80 is applied to the other input of adder 76. Adder 76 and register 80 comprise a “phase accumulator”. They will accumulate the desired speed 56 as if it were a small angle around a circle. When the circle is completed, the adder overflow signal 88 will occur, causing the stepper driver 90 to apply a step to stepping motor 92 via its windings 92. The stepper driver 90 is a micro-stepping current driver such as Allegro Microsystems A3977. As an example, if the adder 76 and register 80 are 20 binary bits, the fast clock rate 82 is 1.048576 MHz and the desired speed 68 is 3000, the adder will overflow every 333.33 microseconds, or precisely 3000 steps per second. Thus the circuit 56 of FIG. 4 synthesizes any frequency (up to the maximum stepping speed of the stepping motors, which is approximately 15,000 steps per second) upon command of the computer 58. The frequency synthesizer 56 could be implemented by discrete logic, but is implemented in a Xilinx Spartan 2 field-programmable-gate-array in the preferred embodiment.

Given the precise control over the elevator and orbit stepping motors, the computer 58 controlling the scanner provides control over the orbit period T in equation 5 to keep the orbit time directly proportional to the breast diameter D, at a constant laser power P and beam diameter d. The control over the orbit period provides the means for reducing the scan time while maintaining the signal quality at the detectors, since the orbit period is decreased as the diameter of the breast at the level of the laser beam and the detectors (scanning plane) is decreased, as the scanning progresses from the chest wall toward the nipple. To maintain the helix angle, the elevator speed will be kept proportional to the orbit speed which will be kept inversely proportional to the breast diameter.

A computer control 95, which provides the means for controlling the power output of a laser with programmable current source, is disclosed in FIG. 5. The computer 58 sends a laser current value to a digital to analog converter 96 over I/O bus 62. The DAC 98 creates an analog setpoint voltage 98 that is proportional to the laser drive current. Operational amplifier 100 amplifies that setpoint voltage and applies it as voltage 102 to the gate of an N-channel FET 104. FET 104, in a source-follower configuration, applies the gate voltage 102, minus 2-3 volts, to its drain as signal 106 and resistor 108, the current sense resistor. Current from FET 104, through resistor 108, forward biases the laser diode 110, causing it to emit light. The laser drive current through resistor 108 creates a small voltage drop, typically less than ½ volt, which is amplified by operational amplifier 112 and resistors 114, 116, 118 and 120. The output 122 of operational amplifier 112, a current sense voltage, is therefore proportional to the laser drive current, for example, 3.0 volts per ampere. This current sense voltage is applied as negative feedback to the operational amplifier 100, therefore stabilizing the loop. The computer control 95 provides the means for adjusting the drive current, and hence the power output of the laser diode 110.

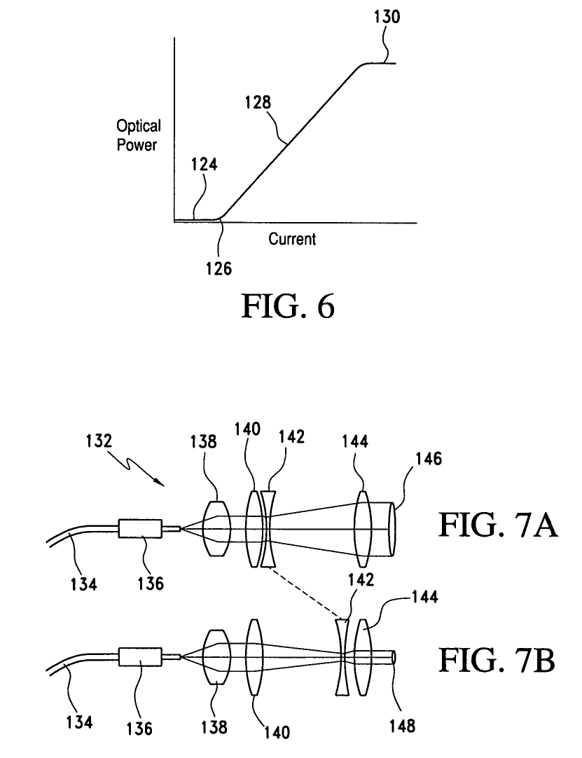

FIG. 6 shows a graph of the laser optical power output versus its drive current, its transfer function. At low currents at region 124, the output power is essentially zero. At a threshold current 126 the laser “turns on” and starts to emit light. Over a wide current range 128, the output power increases linearly with increases in drive current, up to some maximum output level 130, where the laser output can no longer increase.

Based on the transfer function for the laser and the programmable current source of the laser diode 110, the computer 58 controlling the scanner advantageously controls the laser power P in equation 5 to keep the laser power proportional to the breast diameter D, at a constant orbit time T and beam diameter d. The computer control 95 that controls the drive current to the laser provides the means for adjusting the power output of the laser in direct proportion to the diameter of the breast at the scan plane while maintaining the measured signal quality, to account for the decreasing breast diameter at the scan plane as scanning proceeds from near the chest wall toward the nipple and thereby control the temperature rise on the breast surface to an acceptable level.

A variable spot size laser collimator 132 is disclosed in FIGS. 7A and 7B. The laser diode (not shown) is fiber coupled through optical fiber 134 to an optical connector, such as an SMA connector 136. Lens 138 is a collimating lens, which takes the diverging light from the fiber and makes it parallel. Lens 138 is often an aspheric lens with a very short focal length compared to its diameter. The parallel light from lens 138 enters DCX lens 140 which starts the light converging. In FIG. 7A, DCV lens 142 immediately starts the light diverging to DCX lens 144 which returns the light to a parallel beam of a large diameter, spot size 146. In FIG. 7B, DCV lens 142 has been moved near to DCX lens 144 and the beam spot 148 is much smaller. In practice, lens 140 and/or 144 may have to move as lens 142 moves in order to maintain beam parallelness. The movement of the lenses can easily be motorized and controlled by computer 58 (see FIG. 4). This becomes the equivalent of a motorized zoom lens, which is quite common in photography. Thus, the computer 58 controlling the scanner can control the laser spot diameter d in equation 5 to keep the laser spot size inversely proportional to the breast diameter D, at a constant orbit time T and laser power P. It should be understood that the variable spot size laser collimator 132 provides the means for adjusting the laser beam spot diameter in inverse proportion to the diameter of the breast at the scan plane, while maintaining the measured signal quality at the detectors.

It should be understood that the computer 58 can control more than one variable at a time as the breast diameter changes—orbit time, laser power and/or laser spot size according to equation 5. Thus, the control over these variables provides the means for increasing the measured signal at the detectors while controlling the temperature rise on the breast surface.

In the preferred embodiment, the laser is a CW (continuous wave) diode laser operated at 808 nanometer wavelength. Alternative embodiments include other types of lasers, such as solid-state (Ti-sapphire, for example) and time-resolved fast pulse measurements or frequency-domain measurements, all well known in the biomedical optical community.

The preferred embodiment is described with a single laser. Multiple lasers could be employed as disclosed in U.S. Pat. Nos. 6,571,116 and 6,738,658.

The preferred embodiment is described as a third-generation CT geometry, where the laser source and detectors rotate together. Alternatively, the detectors could form a complete stationary ring with just the laser rotating, a fourth-generation CT geometry.

While this invention has been described as having preferred design, it is understood that it is capable of further modification, uses and/or adaptations following in general the principle of the invention and including such departures from the present disclosure as come within known or customary practice in the art to which the invention pertains, and as may be applied to the essential features set forth, and fall within the scope of the invention or the limits of the appended claims.

We claim:

1. An apparatus for breast scanning, comprising:
a) a patient support for a patient to rest in a prone position, said support having an opening with one of her breasts vertically pendent through said opening for scanning;
b) a laser CT scanner disposed below said support for generating data for reconstruction of images of the breast;
c) said laser CT scanner including a laser beam for impinging on the breast, said laser beam being orbitable around the breast;
d) said laser CT scanner including a plurality of detectors positioned in an arc around the breast to simultaneously detect light transmitted through the breast; and
e) means for maintaining the measured signal level at said detectors to an acceptable level while controlling the temperature rise on the breast surface during scanning.

2. An apparatus as in claim 1, wherein said means for maintaining includes means for adjusting the power output of said laser CT scanner.

3. An apparatus as in claim 1, wherein:
a) said laser CT scanner includes a diode laser;
b) said means for maintaining includes means for adjusting the power output of said laser diode in direct proportion to the diameter of the breast at a scan plane.

4. An apparatus as in claim 3, wherein:
a) said laser diode includes a drive current source; and
b) said means for adjusting includes means for adjusting said drive current source.

5. An apparatus as in claim 1, wherein said means for maintaining includes means for adjusting the laser beam diameter in inverse proportion to the diameter of the breast at a scan plane.

6. An apparatus as in claim 1, wherein:
a) said laser beam is orbitable around the breast at time T per each complete orbit; and
b) said means for maintaining includes means for adjusting said time T in direct proportion to the diameter of the breast at a scan plane, thereby allowing for an increased power output of said laser CT scanner while controlling the temperature rise on the surface of the breast to an acceptable level.

7. An apparatus for breast scanning, comprising:
a) a patient support for a patient to rest in a prone position, said support having an opening with one of her breasts vertically pendent through said opening for scanning;
b) a laser CT scanner disposed below said support for generating data for reconstruction of images of the breast;
c) said laser CT scanner including a laser beam for impinging on the breast, said laser beam being orbitable around the breast;
d) said laser CT scanner including a plurality of detectors positioned in an arc around the breast to simultaneously detect light transmitted through the breast; and
e) said laser CT scanner including an adjustable power output in direct proportion to the diameter of the breast at a scan plane, thereby to maintain the measured signal level at said detectors to an acceptable level during scanning.

8. An apparatus as in claim 7, wherein said power output is adjustable in a range of 500 milliwatts to 10 watts.

9. An apparatus as in claim 7, wherein
a) said laser CT scanner includes a laser diode having a drive current source; and
b) means for adjusting said drive current source thereby to adjust said power output.

10. An apparatus for breast scanning, comprising:
a) a patient support for a patient to rest in a prone position, said support having an opening with one of her breasts vertically pendent through said opening for scanning;
b) a laser CT scanner disposed below said support for generating data for reconstruction of images of the breast;
c) said laser CT scanner including a laser beam for impinging on the breast, said laser beam being orbitable around the breast;
d) said laser CT scanner including a plurality of detectors positioned in an arc around the breast to simultaneously detect light transmitted through the breast; and
e) said laser beam having an adjustable laser beam diameter in inverse proportion to the diameter of the breast at a scan plane, thereby to maintain the measured signal level at said detectors to an acceptable level while controlling the temperature rise on the breast surface during scanning.

11. An apparatus as in claim 10, wherein said spot diameter is adjustable over a range of 0.5 millimeter to 5 millimeters.

12. An apparatus as in claim 10, and further comprising a plurality of lenses for enlarging or reducing said spot diameter.

13. An apparatus for breast scanning, comprising:
a) a patient support for a patient to rest in a prone position, said support having an opening with one of her breasts vertically pendent through said opening for scanning;
b) a laser CT scanner disposed below said support for generating data for reconstruction of internal images of the breast;
c) said laser CT scanner including a laser beam for impinging on the breast, said laser beam being orbitable around the breast at time T per each complete orbit;
d) said laser CT scanner including a plurality of detectors positioned in an arc around the breast to simultaneously detect light transmitted through the breast; and
e) said time T is adjustable in direct proportion to the diameter of the breast at a scan plane thereby to maintain the measured signal level at said detectors to an acceptable level while controlling the temperature rise on the breast surface during scanning.

14. An apparatus as in claim 13, wherein said time T is adjustable over a range of 0.2 to 10 seconds.

15. A method for scanning a breast, comprising:
a) positioning a patient in a prone position on a support having an opening with one of her breasts vertically pendent through the opening;
b) scanning the breast with a laser CT scanner with a laser beam orbiting around the breast;
d) detecting with a plurality of detectors positioned in an arc around the breast the light transmitted through the breast;
e) determining the perimeter of the breast; and
f) decreasing the orbit time as the diameter of the breast at scanning planes decreases, thereby reducing the scan time for the breast.

16. A method for scanning a breast with a laser CT scanner having a laser beam for impinging on the breast, comprising:
a) determining the perimeter of the breast being scanned; and
b) adjusting the power level of the laser beam during scanning in direct proportion to the diameter of the breast at a scanning plane.

17. A method as in claim 16, wherein:
a) said laser beam is generated by a laser diode having an adjustable drive current source; and
b) said adjusting is implemented by increasing or decreasing, respectively, the drive current source.

18. A method for a breast with a laser CT scanner having a laser beam for impinging on and orbiting around the breast, comprising:
a) determining the perimeter of the breast being scanned;
b) determining the orbit speed of the laser beam around the breast; and
c) adjusting the orbit speed of the laser beam during scanning in direct proportion to the diameter of the breast at a scanning plane.

19. A method for a breast with a laser CT scanner having a laser beam for impinging on the breast with beam spot, comprising:
a) determining the perimeter of the breast being scanned; and
b) adjusting the beam diameter of the laser beam during scanning in inverse proportion to the diameter of the breast at a scanning plane.
